(19) United States
(12) Patent Application Publication
(10) Pub. No.: US 2024/0424045 A1
(43) Pub. Date: Dec. 26, 2024

(54) BERBERIS COMPOSITION FOR COGNITIVE HEALTH

(71) Applicant: OmniActive Health Technologies Limited, Mumbai, Maharashtra (IN)

(72) Inventors: Muralidhara PADIGARU, Thane (W), Maharashtra (IN); Abhijeet MORDE, Thane (W), Maharashtra (IN); Manutosh ACHARYA, Thane (W), Maharashtra (IN); Sumit PATIL, Thane (W), Maharashtra (IN); Ninad PURANIK, Thane (W), Maharashtra (IN); Anup DESHPANDE, Thane (W), Maharashtra (IN); Munja BAKAN, Thane (W), Maharashtra (IN)

(73) Assignee: OmniActive Health Technologies Limited, Mumbai, Maharashtra (IN)

(21) Appl. No.: 18/566,653

(22) PCT Filed: Jan. 10, 2023
(86) PCT No.: PCT/IB2023/050184

§ 371 Date: Dec. 3, 2023

(30) Foreign Application Priority Data
Feb. 21, 2022 (IN) 202221009121

(51) Int. Cl. A61K 36/29; A61K 31/015; A61K 31/14; A61K 31/355; A61K 31/4525; A61K 45/06; A61P 25/28

(52) U.S. Cl. CPC A61K 36/29; A61K 31/015; A61K 31/14; A61K 31/355; A61K 31/4525; A61K 45/06; A61P 25/28

(57) ABSTRACT

The present invention relates to an oral berberis extract composition and the process for the preparation of the composition of berberis extract as whole or its salts and/or in combination with berberine, jatrorrhizine, and columbamine or its salts with at least one pharmaceutically and/or nutraceutically accepted excipients for prevention, improvement, and maintenance of cognitive health to a subject in need thereof. Further encompassing to the oral berberis composition wherein the berberis extract is comprised of berberis extract as whole or its salts and/or in combination with berberine and/or its salt, and optionally jatrorrhizine, and columbamine or its salts in different forms like powder, beadlets, granules, capsules, tablets, oil suspension, films or any other suitable oral or topical dosage forms. A method of use of the berberis extract composition for prevention, improvement, and maintenance of cognitive health.

### RELATED APPLICATIONS

This application claims priority to the Indian provisional application number IN202221009121, filed on Feb. 21, 2022, which is incorporated herein by reference in its entirety.

### FIELD OF THE INVENTION

The present invention is related to an oral composition comprising Berberis aristata extract and methods for prevention, improvement, and maintenance of cognitive health comprising administering the oral composition to a subject in need thereof. The oral composition, as disclosed herein, improves memory and recall, improves concentration and focus, improves cognitive skills like reasoning and decision-making, improves daily productivity, improves multi-tasking, alertness, and attention, and helps manage stress and increase brain-derived neurotrophic factors (BDNF), a compound associated with learning memory/cognitive function. The oral composition comprises Berberis aristata extract, and at least one pharmaceutically and/or nutraceutically accepted excipients. The Berberis aristata extract may comprise Berberis extract as whole or in combination with active components/constituents selected from berberine, jatrorrhizine, and columbamine and/or its salts. Further, the invention is related to the process of extraction for Berberis aristata extract as a whole or its salts and/or in combination with berberine, jatrorrhizine, and columbamine and/or its salts. Furthermore, the invention is related to the process of preparation of the oral composition of Berberis extract as a whole or its salts, and at least one pharmaceutically and/or nutraceutically accepted excipients for prevention, improvement, and maintenance of cognitive health. The oral composition may comprise Berberis aristata extract as whole or its salts and/or in combination with berberine, jatrorrhizine, and columbamine and/or its salts in a suitable ratio which is formulated in various forms such as powder, beadlets, granules, capsules, tablets, oil suspensions, gummies, chewies, films or any other suitable oral or topical dosage forms using pharmaceutically and/or nutraceutically acceptable excipients.

### BACKGROUND OF THE INVENTION

Cognition refers to a broad range of cognition processes, including psychomotor, attention, memory processes, and executive functions. Recent studies indicate that several negative lifestyle factors can contribute to the development of cognitive impairment; WHO estimated that 47 million people have advanced cognitive impairment worldwide, and the estimation is that this number will increase to nearly 75 million in 2030 and to 132 million in 2050. Mild cognitive impairment (MCI) is regarded as the transitional period between the normal cognitive decline of healthy aging and dementia. Because MCI develops years before dementia and potentially affects individuals younger than 65, there are likely far more than 5 million Americans—of any age—with MCI due to Alzheimer's disease. According to estimations by the Alzheimer's Association, 15-20% of individuals aged 65 and above suffer from mild cognitive impairment. It is well established that individuals who are affected by cognitive decline are at a greater risk of developing dementia, and it has been suggested that early detection and intervention may be an effective preventive strategy.

US20060223838 relates to methods and compositions for treating hyperlipidemia in mammalian subjects. The berberine compound or berberine-related or derivative compound is provided for treating hyperlipidemia, cardiovascular disease, and pulmonary hypertension by administering berberine compound.

IN 7810/DELNP/2011 relates to methods and compositions containing a berberine related compounds are provided for the prevention and treatment of metabolic and cardiovascular disorders, including metabolic syndrome, hyperlipidemia, obesity, diabetes, insulin resistance, hyperglycemia, hypertension, and elevated cholesterol in mammalian subjects by employing berberine and related compounds.

A patent application CHE-2013-06094A relates to a composition for treating cancer. The composition for treating cancer contains berberine. The present invention also relates to a method of preparing the composition and a therapeutically suitable formulation that is to be given to the patients.

Although many health benefits of the berberis extract are known, none of the references focuses on their role in the prevention of cognitive impairment, improvement, and maintenance of cognitive health. Medical advances have improved treatments of numerous diseases, but the cognitive impairment and/or implications have not been sufficiently addressed.

It is well established that individuals who are affected by cognitive decline are at a greater risk of developing dementia and it has been suggested that early detection and intervention may be an effective preventive strategy

Thus, there is a need to have an effective solution for the prevention, improvement, and maintenance of cognitive health, particularly through dietary supplementation, which is convenient for the person in need.

### SUMMARY OF THE INVENTION

In one embodiment described herein, an oral composition comprising Berberis aristata extract is disclosed. The berberis extract comprises one or more berberine, jatrorrhizine, columbamine, and/or a salt thereof.

In one aspect of the embodiment, the oral composition described herein comprises Berberis aristata extract in an amount ranging from 20% to 50% w/w of the composition. In another aspect of the embodiment, the Berberis aristata extract within the oral composition comprises berberine hydrochloride in a range of 5% to 50% w/w of the extract.

In another aspect of the embodiment, the oral composition described herein further comprises at least one pharmaceutically and/or nutraceutically accepted excipient. At least one or more pharmaceutically or nutraceutically acceptable ingredients can be selected from the group consisting of hydrophilic carrier, PGP Inhibitor, Bioenhancer, Binder, emulsifier, pH modifier, and anticaking agent.

In one embodiment, the hydrophilic carrier can be present in a range of 30% to 90% w/w of the composition and can be selected from the group consisting of hydroxypropyl methyl cellulose, microcrystalline cellulose, polyacrylates, polyethylene glycols, povidones, starch and starch derivatives, gums, sugars, plant polysaccharides, and a combination thereof.

In another embodiment, the PGP Inhibitor can be present in a range of 0.1% to 20% w/w of the composition and can be selected from the group consisting of Piperine, Vitamin E TPGS (d-α-Tocopheryl polyethylene glycol 1000 succinate), alkaloids, polysorbates flavonoids, coumarins, resins, saponins, and a combination thereof.

In yet another embodiment, the Bioenhancer can be present in a range of 0.01% to 10% w/w of the composition and can be selected from the group consisting of piperine, d-limonene, monoglycerides, and phospholipids such as lecithin/phosphatidyl choline, glyceryl monostearate botanical extracts, and a combination thereof.

In yet another embodiment, the binder can be present in a range of 0.1% to 15% w/w of the composition and can be selected from the group consisting of Hydroxy propyl methyl cellulose, Methyl cellulose, Hydroxy Propyl cellulose, Polyvinyl Pyrrolidone, Starch, and natural gums such as gum acacia, gum tragacanth, starch, guar gum, and a combination thereof.

In yet another embodiment, the emulsifier can be present in a range of 0.01% to 10% w/w of the composition and can be selected from the group consisting of stearic acid, phosphatidylcholine, polysorbate, sugar alcohols, glycerol & derivatives, and a combination thereof.

In yet another embodiment, the pH modifier can be present in a range of 0.01% to 10% w/w of the composition and can be selected from the group consisting of sodium bicarbonate, citric acid, trisodium citrate, lactic acid, L-arginine, calcium carbonate, magnesium carbonate, and a combination thereof.

In yet another embodiment, the anticaking agent can be present in a range of 0.01% to 5% w/w of the composition and can be selected from the group consisting of silicon dioxide, magnesium stearate, stearic acid, mannitol, and a combination thereof.

In one embodiment, the instant application discloses an oral composition comprising: (i) Berberis aristata extract, wherein the extract comprises Berberine hydrochloride in an amount of 10% to 20% w/w of the extract, (ii) Microcrystalline Cellulose (MCC 101) in an amount of 62% to 68.5% w/w of the composition, (iii) Colloidal Silicon Dioxode (Aerosil) in an amount of 0.5% w/w of the composition, (iv) Sunflower Lecithin (Phosphatidylcholine) in an amount of 1% w/w of the composition, (v) Polysorbate 80 in an amount of 2% w/w of the composition, (vi) Calcium Carbonate in an amount of 4% w/w of the composition, (vii) Hydroxy propyl methyl cellulose (HPMC E3) in an amount of 1.5% w/w of the composition, and (viii) Methacrylic acid ethyl acrylate copolymer (Kollicoat MAE 30DP) in an amount of 2.5% w/w of the composition.

In another embodiment, the instant application discloses an oral composition as described above for use in preventing, improving, and/or maintaining cognitive health. In one aspect of the embodiment, the instant application discloses an oral composition as described above for use in improving memory and recall, improving concentration and focus, improving cognitive skills like reasoning, and decision making, improving daily productivity, improving multi-tasking, alertness, attention and help manage stress and increase brain-derived neurotrophic factor (BDNF, a compound associated with learning memory/cognitive function).

In another embodiment, the instant application discloses a method for preventing cognitive impairment, improving and/or maintaining cognitive health in a subject by comprising administering the oral composition described herein to a subject in need thereof. In one aspect of the embodiment, the instant application discloses a method for improving memory and/or recall in a subject comprising administering the oral composition described herein to a subject in need thereof. In yet another aspect of the embodiment, the instant application discloses a method for improving concentration and/or focus on a subject comprising administering the oral composition described herein to a subject in need thereof. In yet another aspect of the embodiment, the instant application discloses a method for improving cognitive skills, including reasoning and/or decision-making in a subject comprising administering the oral composition described herein to a subject in need thereof. In yet another aspect of the embodiment, the instant application discloses a method for improving daily productivity, multi-tasking, alertness, and/or attention in a subject comprising administering the oral composition described herein to a subject in need thereof. In yet another aspect of the embodiment, the instant application discloses a method for managing stress in a subject comprising administering the oral composition described herein to a subject in need thereof. In yet another aspect of the embodiment, the instant application discloses a method for increasing levels of BDNF in a subject comprising administering the oral composition described herein to a subject in need thereof.

In one embodiment, disclosed herein is a process for preparing Berberis aristata extract as whole or its salts and/or in combination with berberine, jatrorrhizine, and columbamine and/or its salts from berberis plant, more particularly from stems and roots of the plant.

In another embodiment, disclosed herein is a process for the preparation of an oral composition of Berberis aristata extract comprising berberis extract as whole or its salts and/or in combination with berberine, jatrorrhizine, and columbamine and/or its salts in a suitable ratio with pharmaceutically and/or nutraceutically accepted excipients.

In yet another embodiment, disclosed herein is an oral composition of Berberis aristata extract comprising berberis extract as whole or its salts and/or in combination with berberine, jatrorrhizine, and columbamine and/or its salts in a suitable ratio.

In yet another embodiment, disclosed herein is an oral composition of Berberis aristata extract comprising berberis extract as whole or its salts and/or in combination with berberine, jatrorrhizine, and columbamine and/or its salts in a suitable ratio and at least one pharmaceutically and/or nutraceutically accepted excipients.

In yet another embodiment, disclosed herein is an oral composition of Berberis aristata extract as described above formulated in different forms such as orally administrable solid, semisolid, and liquid forms, selected from, but not limited to, dosages such as powders, granules, pellets, beadlets, caplets, tablets, capsules, soft gel capsules, gummies, chewies, solution, emulsions, suspensions, oil suspensions, dispersions and the like.

### BRIEF DESCRIPTION OF THE FIGURES

FIG. 1: Open field test—The Open Field task is a sensorimotor test used to determine general activity levels, gross locomotor activity, and exploration habits in rodent models of CNS disorders. (p<0.005—*/#/$/\hat{ }/↓/↑ by Tukey's multiple comparison test, *—significant with respect to group 1, #—significant with respect to group 2, $ significant with respect to group 3, \hat{ }—significant with respect to group 4, ↓—significant with respect to group 5, ↑—significant with respect to group 6.)

FIG. 2: Morris water maze test—The Morris water maze (MWM) test is used to evaluate behavior linked with the learning and memory of the rats. (p<0.005—*/#/$/\hat{ }/↓/↑/ [symbol] by Tukey's multiple comparison test, *—significant with respect to group 1, #—significant with respect to group 2, $ significant with respect to group 3, \hat{ }—significant with respect to group 4, ↓—significant with respect to group 5, ↑—significant with respect to group 6.)

FIG. 3: Novel object recognition test—The Novel object recognition (NOR) test is used to evaluate cognition, particularly recognition memory, in rodent models of CNS disorders. (p<0.005—*/#/$/\hat{ }/↓/↑ by Tukey's multiple comparison test, *—significant with respect to group 1, #—significant with respect to group 2, $ significant with respect to group 3, \hat{ }—significant with respect to group 4, ↓—significant with respect to group 5, ↑—significant with respect to group 6, n=7.)

FIG. 4: Y-maze test—The Y-maze test is widely used for the evaluation of working memory (or short-term memory). (p<0.005—*/#/$/\hat{ }/↓/↑ by Tukey's multiple comparison test, *—significant with respect to group 1, #—significant with respect to group 2, $ significant with respect to group 3, \hat{ }—significant with respect to group 4, ↓—significant with respect to group 5, ↑—significant with respect to group 6, [symbol] —significant with respect to group 7, n=7.)

FIG. 5: Evaluation of neurotropic factor-brain-derived neurotropic factor (BDNF). (Values are expressed in Mean and SD, *—significant with respect to G1, #—significant with respect to G2. */#—p<0.005, **/##—p<0.01, ***/###—p<0.001.)

FIG. 6: Outline of experimental design for the study—This test describes the overall experiment outline.

### DETAILED DESCRIPTION OF THE INVENTION

The present invention mainly encompasses the Berberis aristata extract composition, a process for preparation, and its use in cognitive health wherein the berberis composition consists of berberine and/or its salt as a major constituent, which is an alkaloid, organic heteropentacyclic compound, which is found in many deciduous, and evergreen shrubs found in temperate and subtropical climates. Berberine is quaternary ammonium that can be synthesized from said plant extracts. One such plant is Berberis aristata. The most common form of berberine salt is berberis hydrochloride or berberine chloride.

The present invention is to provide the oral composition of Berberis aristata extract comprising berberis extract as whole or its salts and/or in combination with berberine, jatrorrhizine, and columbamine and/or its salts from the parts of the berberis plant. Wherein the berberis extract as a whole consists of berberine or its suitable salt and/or combination with other constituents such as jatrorrhizine and columbamine. The Berberis aristata extract is extracted from the berberis plant, mainly from the stems and roots of the plant.

Further to this present invention is described herein relates to an oral composition of berberis as defined above and used for prevention, improvement, and maintenance of the cognitive health category. More particularly, the present invention is provided using the oral berberis composition, which improves memory and recall, improves concentration and focus, improves cognitive skills like reasoning and decision making improves daily productivity, improves multi-tasking, alertness, and attention, and helps manage stress and increases BDNF, a compound associated with learning memory/cognitive function.

The terminology “oral composition and/or oral berberis composition and/or Berberis aristata extract composition” from Berberis aristata extract is commonly used in the specification to refer to a composition having berberis extract as a whole and/or its salt in selectively added active components/constituents such as berberine, jatrorrhizine, and columbamine or its salts with one or more pharmaceutically and/or nutraceutically accepted excipients. But more preferably, berberis composition comprises berberine or its suitable salt, such as hydrochloride in a preferred ratio suitable for berberis composition and used for the cognitive health category.

It is to be appreciated that the term “Berberis aristata extract” can be interpreted to be within the scope of, which in general includes Berberis aristata extract as a whole, including berberine or its suitable salts and/or in combination with jatrorrhizine and columbamine and/or its salts.

The term herein referred “cognitive health” means cognitive health, which is the ability to clearly think, learn, and remember. It is an important component of brain health. Others include motor function, which is how well a person can move and control their movements; emotional function, which is how well a person can interpret and respond to emotions; and sensory function, which evaluates how well a person can feel and respond to sensations of touch, such as pressure, pain, and temperature.

Cognitive reserve is the mind's resistance to damage to the human brain. It indicates a resilience to neuropathological damage. Cognitive reserve refers to the capacity of a person to meet life's cognitive demands. This capacity is evident in an ability to assimilate information, comprehend relationships, and develop reasonable conclusions and plans. The emphasis here is in the way the brain uses its damaged resources. It could be defined as the ability to optimize or maximize performance.

It is to be appreciated that the term “Berberis aristata extract” as a whole can be interpreted to be within the scope of, which in general includes berberine and/or its salt either alone or in combination with jatrorrhizine and/or columbamine and/or its salts.

It is to be appreciated that the term “salts” can be interpreted to be within the scope of, which in general includes suitable salts like hydrochloride/chlorides, acetate, phosphates, more particularly hydrochlorides such as berberine hydrochloride, and optionally jatrorrhizine hydrochloride, and columbamine hydrochloride.

According to an embodiment of the present invention, oral compositions of Berberis aristata extract the invention are comprised of the berberis extract as whole or its salts and/or in combination with berberine, jatrorrhizine, and columbamine and/or its salts also one or more of pharmaceutically and/or nutraceutically accepted excipients.

In berberis composition, berberine and/or its salt is a major active ingredient/constituent along with optionally jatrorrhizine and columbamine and/or its salts extracted from Berberis aristata plant, also known as Indian barberry, is a shrub belonging to the family berberidaceae and the genus berberis. Berberine is also widely present in barks, leaves, twigs, rhizomes, roots, and stems.

According to a further embodiment, the aqueous solvent can be selected from water, phosphoric acid, hydrochloric acid, or a combination thereof. Also, optionally an organic solvent can be used selected from methanol, ethanol, propanol, butanol ethyl acetate, acetone, hexene, and chloroform or a combination thereof.

According to a further embodiment, the berberis extract comprises berberine hydrochloride not less than 50% w/w of the total extract.

According to a further embodiment, the berberis extract comprises berberine hydrochloride not less than 40% w/w of the total extract.

According to a further embodiment, the berberis extract comprises berberine hydrochloride not less than 30% w/w of the total extract.

According to a further embodiment, the berberis extract comprises berberine hydrochloride not less than 20% w/w of the total extract.

According to a further embodiment, the berberis extract comprises berberine hydrochloride not less than 10% w/w of the total extract.

According to a preferred embodiment, the Berberis aristata extract comprises berberine and/or its salt, such as hydrochloride is present in not less than 10-20% w/w of the total extract.

According to a further embodiment, the berberis extract comprises jatrorrhizine and/or its salts not less than 1% w/w of the total extract.

According to a further embodiment, the berberis extract comprises jatrorrhizine and/or its salts not less than 2% w/w of the total extract.

According to a further embodiment, the berberis extract contains columbamine and/or its salts not less than 0.01% w/w of the total extract.

According to a further embodiment, the berberis extract contains columbamine and/or its salts not less than 0.1% w/w of the total extract.

Oral compositions/berberis composition described herein are comprised of Berberis aristata extract as whole or its salts and/or in a combination of its active components/constituents selected from berberine and/or its salt and optionally, jatrorrhizine and columbamine or its salts and/or in at least one or more pharmaceutically and/or nutraceutically acceptable excipients.

More preferably, oral berberis compositions as defined above are formulated using suitable excipients selected from the group of, but not limited to, solvents, bioavailability enhancers, PGP inhibitors, pH modifiers, emulsifiers, carriers, anticaking agents, coating agents, and oils or the combination thereof.

According to further embodiment oral berberis compositions described herein can be available in orally administrable solid, semisolid, liquid forms, selected from, but not limited to dosages such as, powders, granules, pellets, beadlets, caplets, tablets, capsules, soft gel capsules, solution, emulsions, suspensions, oil suspensions, dispersions and the like.

Oral berberis compositions, as described herein, are comprised of Berberis aristata extract as whole or its salts and/or in combination with berberine and/or its salts and optionally jatrorrhizine, and columbamine and/or its salts with at least one excipient such as pharmaceutically and/or nutraceutically accepted excipients which can be formulated as granules, powder, oil suspensions and/or beadlets.

In some more embodiments, a process for the preparation of an oral berberis composition is in the form of powder comprises:
(i) Taken a solvent in a clean vessel;
(ii) Added bioenhancer, PGP Inhibitor, pH modifier and emulsifier under stirring to obtained clear solution and heat the solution at 50-65° C.;
(iii) Added berberis extract containing berberine hydrochloride to the solution obtained in step (ii) under stirring and stop heating once a clear solution observed;
(iv) Added carrier to a solution, obtained in step (iii) under stirring; and
(v) Spray dried solution obtained from step (iv) to get powder.

In some embodiments, a process for the preparation of oral berberis composition in the form of beadlets comprises:
(i) Prepared a solution of Bioenhancer, PGP Inhibitor, and emulsifier;
(ii) Added berberis extract contains berberine hydrochloride, pH modifier, anticaking agent, and stir to form a uniform blend;
(iii) Added solution prepared in step (i) in the dry blend of step (ii) obtained the wet mass;
(iv) Passed the wet mass through the extruder and Collected the Extrudes for the spheronization process;
(v) Loaded the Extrudes on the pitch spheronization plate to get the spherical beadlets;
(vi) Dried the beadlets in a tray dryer to get the required LOD; and
(vii) Sifted the beadles using vibro-sifter to obtain desired particle size.

In some embodiments, a process for the preparation of an oral berberis composition is in the form of oil suspension comprises:
(i) Added oils, bio-enhancers, PGP inhibitors, and emulsifiers by stirring in a vessel and heating the mixture at 50-65° C.; and
(ii) Added berberis extract contains berberine hydrochloride to step (i) under stirring to obtain oil suspension.

In some preferred embodiments, a process for the preparation of oral composition in the form of granules comprises:

(i) Prepared a solution of Bioenhancer, Binder, PGP Inhibitor, and emulsifier.

(ii) Prepared a solution of polymer;

(iii) Added berberis extract, pH modifier, and anticaking agent and stirred to form a uniform blend;

(iv) Added solutions prepared in steps (i) and (ii) in the dry blend of step (iii) to obtain the wet granules; and

(v) Dried and sifted the granules obtained in step (iv).

In one more embodiment, the solvent used in the preparation of oral berberis composition is selected from the group such as, but not limited to, isopropyl alcohol, acetone, methanol, ethyl acetate, ethanol, methylene dichloride, water, and/or combination thereof.

In one more embodiment, the bioavailability enhancer employed in the preparation of oral berberis compositions is selected from the group such as, but not limited to piperine, d-limonene, monoglycerides, and phospholipids such as lecithin/phosphatidyl choline, glyceryl monostearate botanical extracts and combination thereof.

In one of the embodiments, the PGP inhibitor in preparation of oral berberis composition is selected from a group such as, but not limited to Piperine, Vitamin E TPGS (d-α-Tocopheryl polyethylene glycol 1000 succinate), alkaloids, polysorbates flavonoids, coumarins, resins, saponins and/or combination thereof.

In one more embodiment, the pH modifier in preparation of oral berberis composition is selected from the group such as, but not limited to sodium bicarbonate, citric acid, trisodium citrate, lactic acid, L-arginine, calcium carbonate, magnesium carbonate, and/or combination thereof.

In one more embodiment, a binder used in the preparation of oral berberis composition is selected Hydroxy propyl methyl cellulose, Methylcellulose, Hydroxy Propyl cellulose, Polyvinyl Pyrrolidone, Starch, and natural gums such as gum acacia, gum tragacanth, starch, guar gum and/or a combination thereof.

In one more embodiment, the emulsifier in the preparation of oral berberis composition is selected from the group such as, but not limited to, stearic acid, phosphatidylcholine, polysorbate, sugar alcohols, glycerol & derivatives, and/or combination thereof.

In one more embodiment, the carrier employed in the preparation of oral berberis compositions is selected from the group such as, but not limited to cellulose derivatives, like hydroxypropyl methylcellulose, microcrystalline cellulose, polyacrylates, polyethylene glycols, povidones, starch and starch derivatives, gums, sugars, plant polysaccharides and/or a combination thereof.

In one more embodiment, the anticaking agent in the preparation of oral berberis composition is selected from the group, such as, but not limited to, silicon dioxide, magnesium stearate, stearic acid, mannitol, and/or a combination thereof.

In one more embodiment, the coating agent in the preparation of oral berberis composition is selected from the group such as, but not limited to lipids, waxes, stearic acid, glyceryl di-stearate, shellac, zein, acrylate polymer, cellulose polymer and/or combination thereof.

According to a further embodiment of the present invention, carrier/vehicle may be selected from but not limited to sunflower oil, coconut oil, corn oil, cottonseed oil, canola oil, olive oil, palm oil, peanut oil, safflower oil, sesame oil, soybean oil, rapeseed oil and medium-chain triglyceride (MCT) oil or the combination thereof.

A further embodiment of the present invention is an oral composition of berberis comprising berberine hydrochloride and optionally in combination with jatrorrhizine and columbamine or its salts used for prevention, improvement, and maintenance of cognitive health. More specifically, it improves memory and recall, improves concentration and focus, improves cognitive skills like reasoning, and decision making, improves daily productivity, improves multi-tasking, alertness, and attention, helps manage stress, and increases BDNF (a compound associated with learning memory/cognitive function).

### EXAMPLES

The best mode of performing the invention is illustrated in the examples below. However, the invention is not limited to the examples, and the examples are only meant for the enablement of a person skilled in the art.

### Examples of Granules Preparation

### Example 1

The bioenhancer, binder, and PGP inhibitors/emulsifier were weighed accurately and added to the purified water/appropriate solvent under stirring to prepare a solution. The polymer was weighed accurately and added to an appropriate solvent to prepare the polymer solution. Berberis aristata extract, pH modifier, and anticaking agent were weighed accurately and loaded in an RMG container and dry mixed at appropriate impeller speed to form a uniform blend. The binder and polymer solutions were added gradually to the dry blend in RMG to obtain wet mass/wet granule. Wet granules were then dried and sifted using a sifter.

| | Functional | % | Qty. |
|---|---|---|---|
| Ingredients | Role | W/W | (Kg) |
| Berberis aristata Extract (Berberine HCl | Active | 27.37 | 0.273 |
| salt contain 16.42%) | | | |
| Microcrystalline Cellulose (MCC 101) | Carrier/Diluent | 61.13 | 0.611 |
| Colloidal Silicon dioxode (Aerosil) | Anticaking | 0.50 | 0.005 |
| | agent | | |
| Sunflower Lecithin (Phosphatidylcholine) | Bioenhancer | 1.00 | 0.010 |
| Polysorbate 80 | Emulsifier/PGP | 2.00 | 0.020 |
| | inhibitor | | |
| Calcium Carbonate | pH Modifier | 4.00 | 0.040 |
| Hydroxy propyl methyl cellulose (HPMC | Binder | 1.50 | 0.015 |
| E3) | | | |
| Methacrylic acid ethyl acrylate copolymer | Enteric | 2.50 | 0.025 |
| Kollicoat MAE 30DP) | Polymer | | |
| Water | Solvent | — | Q.S. |

### Example 2

| | Functional | % | Qty. |
|---|---|---|---|
| Ingredients | Role | W/W | (Kg) |
| Berberis aristata Extract (Berberine HCl | Active | 27.37 | 0.273 |
| salt contain 16.42%)) | | | |
| Microcrystalline Cellulose (MCC 101) | Carrier/Diluent | 61.13 | 0.611 |
| Colloidal Silicon dioxode (Aerosil) | Anticaking | 0.50 | 0.005 |
| | agent | | |
| Sunflower Lecithin | Bioenhancer | 1.00 | 0.010 |
| Vitamin E TPGS | Emulsifier | 2.00 | 0.020 |
| Calcium Carbonate | pH Modifier | 4.00 | 0.040 |
| Hydroxy propyl methyl cellulose (HPMC | Binder | 1.50 | 0.015 |
| E3) | | | |
| Methacrylic acid ethyl acrylate copolymer | Enteric | 2.50 | 0.025 |
| | Polymer | | |
| Water | Solvent | — | Q.S. |

Note:
Example 2 was carried out in the same procedural way as Example 1.

### Example 3

### Examples of Beadlets Preparation

The bioenhancer, PGP inhibitors, and emulsifier were weighed accurately and added to the purified water/appropriate solvent under stirring to prepare a solution. Berberis aristata extract and anticaking agent were weighed accurately and loaded in an RMG container and dry mixed at appropriate impeller speed to form a uniform blend. The binder solution was added gradually to the dry blend in RMG to obtain wet mass/wet granule. The wet mass was then passed through an extruder to obtain extrudes. Extrudes were loaded on a pitch spheronizer plate to obtain spherical beadlets/pellets. Spherical beadlets were loaded in a tray dryer for drying to obtain desi % LOD. Dried beadlets were sifted to obtain desired particle size distribution.

| | Functional | % | Qty. |
|---|---|---|---|
| Ingredients | Role | W/W | (Kg) |
| Berberis aristata Extract (Berberine HCl | Active | 27.37 | 0.278 |
| salt contain 16.42%)) | | | |
| Microcrystalline Cellulose (MCC 101) | Carrier/Diluent | 54.13 | 0.541 |
| Colloidal Silicon Dioxode (Aerosil) | Anticaking agent | 0.50 | 0.005 |
| Sunflower Lecithin | Bioenhancer | 1.00 | 0.010 |
| (Phosphatidylcholine) | | | |
| Polysorbate 80 | Emulsifier | 2.00 | 0.020 |
| Xanthan gum | PGP inhibitor | 15.00 | 0.150 |
| Water | Solvent | — | Q.S. |

### Example 4

| | Functional | % | Qty. |
|---|---|---|---|
| Ingredients | Role | W/W | (Kg) |
| Berberis aristata Extract (Berberine HCl | Active | 27.37 | 0.278 |
| salt contain 16.42%)) | | | |
| Microcrystalline Cellulose (MCC 101) | Carrier/Diluent | 66.63 | 0.666 |
| Colloidal Silicon Dioxode (Aerosil) | Anticaking agent | 0.50 | 0.005 |
| Sunflower Lecithin | Bioenhancer | 1.00 | 0.010 |
| Phosphatidylcholine) | | | |
| Polysorbate 80 | Emulsifier/PGP | 2.00 | 0.020 |
| | inhibitor | | |
| Hydroxy propyl methyl cellulose | Binder | 2.50 | 0.025 |
| (HPMC E3) | | | |
| Water | Solvent | — | Q.S. |

### Example 5

### Example of Powder Preparation

The solvent was weighed accurately in a clean vessel and maintained at 50-65° C. by heating. To it accurately weighed, quantities of PGP inhibitors/emulsifiers were added under stirring to obtain a clear solution. Weighed quantity of berberis extract was added to the solution and maintained at 50-65° C. under stirring. Heating was stopped once a clear solution was obtained; Solution was spray dried to obtain a free-flowing powder.

| | Functional | % | Qty. |
|---|---|---|---|
| Ingredients | Role | W/W | (Kg) |
| Berberis aristata Extract (Berberine | Active | 48.00 | 0.480 |
| HCl salt contain 28.8%) | | | |
| Hydroxy propyl methyl cellulose | Binder | 50.00 | 0.500 |
| (HPMC E3) | | | |
| Polysorbate 80 | Emulsifier/PGP | 2.00 | 0.020 |
| | inhibitor | | |
| Water | Solvent | — | Quantity |
| | | | supplied |
| | | | (Q.S.) |
| Ethanol | Solvent | — | Quantity |
| | | | supplied |
| | | | (Q.S.) |

### In-Vitro/In-Vivo Study:

The present study was to assess the efficacy of the oral composition of berberis extract composition comprising berberine hydrochloride in a suitable ratio optionally with jatrorrhizine, and columbamine or columbamine its salts for prevention, improvement, and maintenance of cognitive health. More specifically, it improves memory and recall, improves concentration and focus, improves cognitive skills like reasoning, and decision making, improves daily productivity, improves multi-tasking, alertness, and attention, and helps manage stress and increase BDNF (a compound associated with learning memory/cognitive function).

### Study Objective

The objective of this study was to evaluate the neuroprotective potential therapeutic effects of Berberis extracts on scopolamine-induced cognitive deficits in rats. Berberine is an iso-quinoline alkaloid in the berberis genus with antioxidant and anti-inflammatory properties and protective effects in neurodegenerative disorders.

Scopolamine, which is a muscarinic antagonist drug, blocks presynaptic muscarinic acetylcholine receptors turning to activate dopamine neurons. In acute dosage, scopolamine shows toxic properties on the central nervous system and leads to injury in hippocampal circuits that result in cognitive and memory impairment. Therefore, berberis extract, granules, powder, oil suspension, and beadlets as a composition which was prepared as per the examples provided in the examples section, were evaluated for its ability to mitigate cognitive deficits using various behavioral tests such as open field test, Morris water maze test, Y-maze and novel object recognition test as compared to a known neuroprotective agent citicoline. The various preparations were also evaluated for cytoprotective potential as well as biochemical parameters such as brain-derived neurotrophic factor (BDNF) that regulates synaptic plasticity and cognitive function. Berberine composition treatment has been shown to mitigate alterations in BDNF, a compound associated with learning memory/cognitive function levels in the brain of cognition-impaired rats.

### Material and Methods

Evaluating the neuroprotective potential of the berberis extract by using various behavioral, biochemical, and neurochemical parameters.

### Experimental Animals:

Animals and ethical approval: Male Sprague Dawley rats (weight-180-220 g) were kept at a temperature of 23° C.±2° C. and relative humidity of 50±10%. The 12 hrs. light and dark cycles will be maintained at this temperature and humidity. Animals will be kept for 1 week in animal house facilities for habituation before the start of the experiment. All the procedures were executed as per the approved protocol from the Institutional Animal Ethics Committee.

### Induction of Cognitive Impairment in Rats:

Cognitive impairment/amnesia was induced with scopolamine (3.0 mg/kg, i.p.), and the training trials were performed 15 minutes after injection except in the normal control group

### Treatment Group:

Animals were Randomly Divided into Seven Groups (n=14).

Group 1 (G1) —Normal Control (NC) —This group of animals treated only with vehicle as a carboxy methyl cellulose 0.5% in water.

Group 2 (G2) —Disease Control (DC) animals received vehicle (a carboxy methyl cellulose 0.5% in water.) —This group of animals was treated with the drug scopolamine to induce cognition effect but not treated with Berberis aristata composition to see disease phenotype.

Group 3 (G3) —Animals received Berberis aristata extract 50% (Berberine HCl) 100 mg/kg, —This group was treated with Berberis aristata (Unformulated) extract.

Group 4 (G4) —Animals received Berberine hydrochloride powder, a market sample from—ToniiQ used for cognition at a dosage of 100 mg/kg dose—oral. This group of animals was treated with a marketed sample as provided above.

Group 5 (G5) —Animals received Reference standard-Citicoline at 26 mg/kg-oral, —a marketed drug used for cognition. This group of animals was treated with citicoline at 26 mg/kg.

Group 6 (G6) —Animals received Berberis aristata composition in granules form 15% (dose—100 mg/kg) oral.

Group 7 (G7) —Animals received Berberis aristata composition in granules form 15% (dose—3 mg/kg) oral.

### Procedures:

On day 30 of the treatment, 15 minutes before the behavioral tests, scopolamine was administered at 3.0 mg/kg through the intraperitoneal route (i.p.) to all the groups except normal control animals (G1 received normal saline through i.p.)

Termination procedure: At the end of the study, Blood collection from all the surviving animals was done. All study animals were euthanized and brain from all animals were collected. Serum samples were analyzed for biochemical parameters (AST, ALT, BUN and BDNF)

### Behavioral Parameters:

After 15 minutes of scopolamine injection i.p, seven animals from each group were assessed for two behavioral tests. Behavioral tests viz., Y-maze, Open Filed Test, Morris Water Maze (Escape Latency) and Novel Object Recognition Test were evaluated. All the animals were trained prior to behavioral assessment. Assessment of the treatment effect of extracts on cognition was done on day 30.

TABLE 1
Animal grouping and treatments*

| | | | Duration | No. of |
|---|---|---|---|---|
| Groups | Dose Groups | Route | (in days) | animals |
| G1 | Normal Control + Vehicle | Oral | 33 | 14 |
| G2 | Disease Control + Vehicle | Oral | | 14 |
| G3 | Unformulated Berberine chloride | Oral | | 14 |
| | extract 50% (equivalent to | | | |
| | 100 mg/kg berberine content) | | | |
| G4 | Marketed Berberine hydrochloride | Oral | | 14 |
| | powder (equivalent to 100 mg/kg | | | |
| | berberine content) | | | |
| G5 | Reference standard - Citicoline | Oral | | 14 |
| | (26 mg/kg) | | | |
| G6 | Berberis aristata composition | Oral | | 14 |
| | granules 15% (100 mg/kg) | | | |
| G7 | Berberis aristata composition | Oral | | 14 |
| | granules 15% (3 mg/kg) | | | |

*Note:
Animals of group 2 to group 7 received Scopolamine at 3 mg/kg, intraperitoneal route (i.p.) on day 30 for behavioral parameters assessment.

### Behavioral Tests:

### 1. Open-Field Test

The Open Field task is a sensorimotor test used to determine general activity levels, gross locomotor activity, and exploration habits in rodent models of CNS disorders. Animals were trained for this test 1 day before the actual test. The open field apparatus was an opaque acrylic cage (100×100 cm) with walls 40 cm in height, where the floor was divided with white lines by 16 squares (18×18 cm) of the identical dimension. The entire room, except the Open field, was kept dark during the experiment. The behavioral activity of rats in the open field was observed, and the parameters were recorded as follows: (a) time spent on the Peripheral square, (b) a total number of crossings, (c) central area crossings, and (d) time spent in a central area.

### Conclusion:

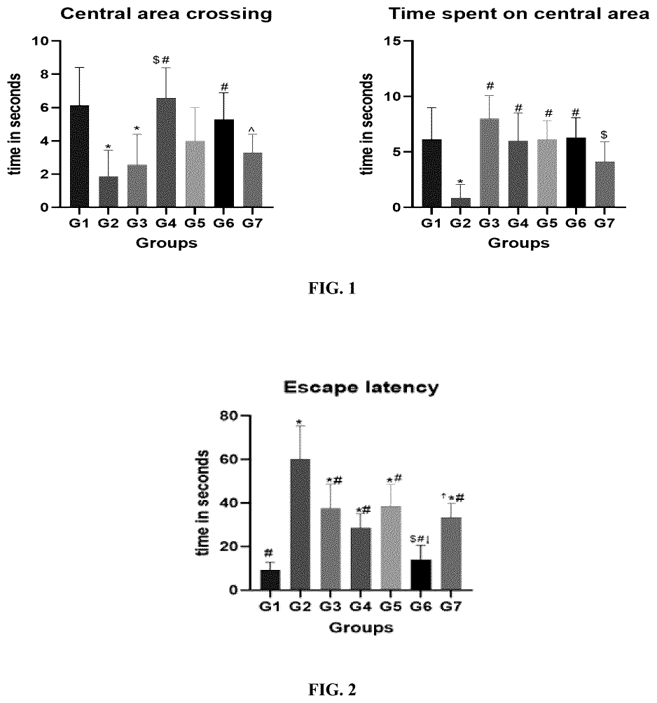

The obtained result from the open field test as per FIG. 1 demonstrated that scopolamine significantly reduced the number of crossing and total time spent in the center of the arena, whereas berberis extract BE (G3) treatment significantly restored the tendency of animals similar to a normal control group (G1). Berberis aristata composition at 100 mg/kg and 3 mg/kg (G6 & G7 respectively) demonstrated superior effect to citicoline (G5) and BE (generic) (G4). The activity is shown in G7 considering the dose of 3 mg/kg, which is significantly low and demonstrates the equivalent effect to the others. Hence, it's concluded that group G7 results in surprising efficacy due to high bioavailability and other related factors.

### 2. Morris Water Maze Test

The Morris water maze (MWM) test is used to evaluate behavior linked with the learning and memory of the rats. Morris water maze consists of the circular pool (160 cm diameter and 50 cm height) filled up to 42 cm with water. The temperature in the pool was maintained at 250 C±10 C. A platform of 12 cm diameter was placed 1 cm below the surface of water. The rats were trained to navigate the submerged platform. The rats were given a maximum time of 120 s (cut-off time) to find the hidden platform and were allowed to stay on it for 30 seconds. The platform remained in the same position during the training days. Rat that failed to locate the platform within 120 seconds was put on platform only in the first session.

Animals were placed in the pool, and were allowed to swim freely. Swimming traces of animals were recorded until they reached the hidden platform and the time length was defined as latency. Escape latency of each rat was monitored and analyzed. Latency to escape the water was considered a measure of learning ability. After 4 days of training, the platform will be removed, and the rats will be allowed to swim freely for 120 sec. The time spent in target quadrant was considered to be an indicator of memory retention.

### Conclusion:

The obtained result from the Morris water maze test as per FIG. 2, scopolamine treatment significantly increased the latency time as compared to the control group (G1), indicating scopolamine-induced memory impairment. However, treatment with BE (G3) significantly reduced the latency time as compared to the scopolamine group, and with BE generic (G4), further improvement and totally abolishing the scopolamine effect. Berberis aristata composition at 100 mg/kg (G6) demonstrated significantly improved performance as compared to unformulated BE (G3) or citicoline (G5), indicating the superiority of formulated products (G6 & G7) in improving spatial learning and memory.

### 3. Novel Object Recognition Test

The Novel Object Recognition (NOR) test is used to evaluate cognition, particularly recognition memory, in rodent models of CNS disorders. Rats were acclimatized over 5 days to the behavioral arena and two identical objects. The object recognition test involved two phases. One phase is a sample phase, and another one is a test phase, both of these phases having 5 minutes duration, during which the time spent by the animal to explore each object was noted. In the sample phase, rats explored two identical objects for 5 min. Subsequently, the test phase was after 1 hour, for which one object will be exchanged with a novel one. The discrimination ratio will be calculated as novel object interaction/total interaction with both objects.

### Conclusion:

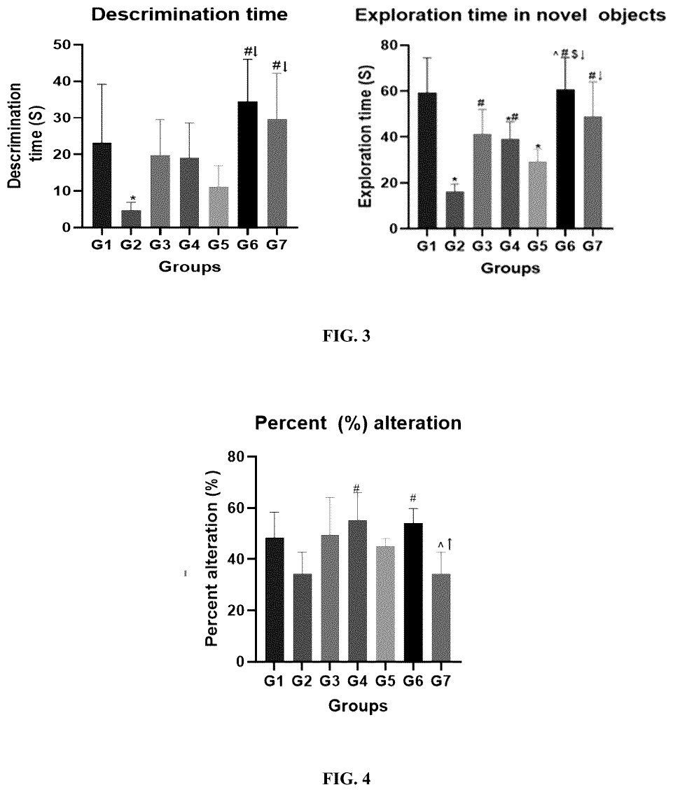

The obtained result from The Novel Object Recognition (NOR) test as per FIG. 3, preference for the novel object in the control group (G1) was significantly reduced by scopolamine measured as a recognition index. BE (50% and Generic—G3 & G4) significantly restored the recognition index, which was further enhanced by Berberis aristata composition products at 100 mg/kg and 3 mg/kg dose (G6 & G7) that showed enhanced learning and memory performance compared to the G3 & G4 and citicoline (G5).

### 4. Y Maze Test:

The Y-maze test is widely used for the evaluation of working memory (or short-term memory). Animals were trained for this test for 3 days of free exposure/exploration of the apparatus before the actual test. The Y-maze apparatus consists of three arms with an angle of 120° between each of the two arms. Each arm is 8 cm wide, 50 cm long, and 20 cm deep. The arms were designated as the start arm (A), novel arm with food stimuli (B), and another arm (C). In the first trial, the rat was placed in the start arm and allowed to move freely through the apparatus for 5 min. In the second trial, the rat was placed in the maze and allowed to explore arms A, B, and C, rats comfortably entered each arm. The ability to alternate requires that the rat knows which arm they have already visited (memory)/the promise of a reward stimulus such as feed. A total of 13 entries were recorded for each rat visually, and finally, the percentage of spontaneous alteration was calculated by using the following formula:

$$Percent ( \% ) alteration = \frac{Number of positive entries \times 100}{( Total number of arm entries - 2 )}$$

### Conclusion:

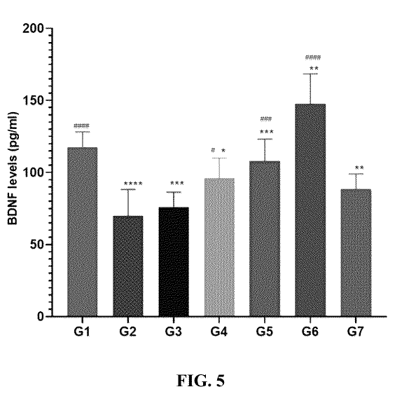

The obtained result from the Y-maze test, as per FIG. 4, shows that BE significantly increased the spontaneous alternation in rats, suggesting improvement of working memory. A Berberis aristata composition of 100 mg/kg & 3 mg/kg dose (G6 & G7) showed a comparably enhancing effect compared to citicoline (G5), BE (50%, and Generic-G3 & G4).

### Evaluation of Neurotropic Factor-Brain-Derived Neurotropic Factor (BDNF)

The synergistic interactions between neuronal activity and synaptic plasticity by BDNF make it an ideal and essential regulator of cellular processes that underlie cognition and other complex behaviors. BDNF plays a critical role in hippocampal long-term potentiation (LTP), a long term enhancement of synaptic efficacy thought to underlie learning and memory. Deficits in BDNF signaling contribute to the pathogenesis of several major diseases and disorders such as Huntington's disease, Alzheimer's disease, and depression. To test this, brain tissue was collected and lysed in phosphate buffer saline (PBS) buffer. The lysates were incubated for 30 min on ice, centrifuged for 20 min at 4000 rpm at 4° C., and the supernatant was collected. Proteins of lysates were quantified using a bicinchoninic acid (BCA) assay kit Sigma-Aldrich (St Louis, MO, USA) followed by quantitative determination of BDNF (a compound associated with learning memory/cognitive function) done using ELISA assay.

### Conclusion:

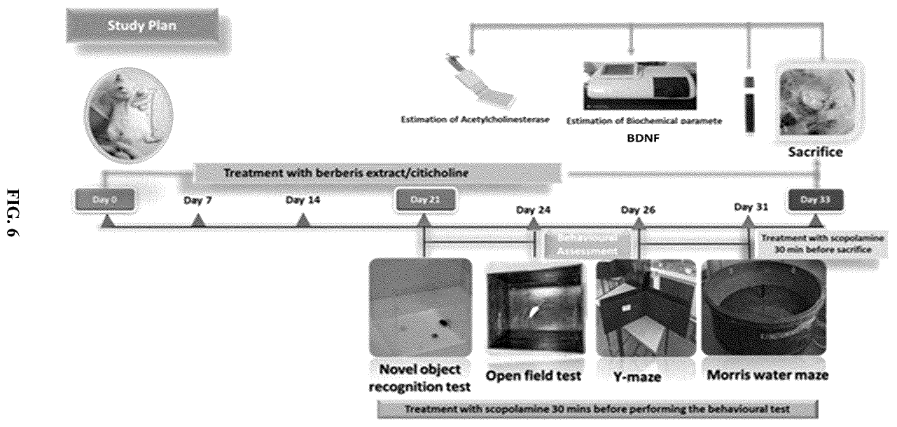

The obtained result from the assay, as per FIG. 5, shows that berberis extract (Generic) significantly increased BDNF levels in the serum of the rats, suggesting that this increase might be responsible for the improved performance of the rats in the behavioral tests of working memory and cognition. The Berberis aristata composition 100 mg/kg dose (Granules-100) showed a better BDNF-enhancing effect compared to citicoline and Berberis aristata composition 3 mg/kg dose (Granules-3).

### Biochemical and Neurochemical Parameters

After the treatment, animals were euthanized, and brain tissue was isolated for the evaluation of biochemical parameters.
Evaluation of organ function test parameters like SGPT, SGOT, BUN, etc.
Evaluation of acetylcholine esterase enzyme

The acetylcholinesterase (AChE) activity was assayed following an adaptation of the spectrophotometric method reported by Ellman et al. The cuvette used as a blank to control for the nonenzymatic hydrolysis of acetylcholine contained a mixture of 500 μL of 3 mM DTNB solution (in 0.1 M potassium phosphate pH 8), 100 μL of 15 mM AChI (in water), 275 μL of 0.1 M potassium phosphate pH 8, and 100 μL of berberine extract solutions at the different concentrations evaluated. In the reaction cuvette, 25 μL of buffer was replaced by AChE solution 0.16 U/mL. The resulting solutions were placed in a spectrophotometer. The thiocholine formed during the hydrolysis of acetylcholine reacts rapidly with DTNB and a yellow compound is formed. The reaction was monitored for 5 min at 405 nm and the absorbance registered every minute. Velocities of reaction were calculated. Enzyme activity was calculated as a percentage of the velocities compared to that of the assay using buffer solution instead of inhibitor. The assays were performed in triplicate.

### Evaluation of Acetylcholine Esterase Enzyme:

We evaluated acetylcholine esterase inhibition activity of the berberis extract in-vitro. Decrease in brain acetylcholine (ACh) levels is implicated in the pathophysiology of cognitive dysfunction. Acetylcholine plays an important role in memory function and has been implicated in aging-related dementia, in which the impairment of hippocampus-dependent learning strongly manifests. AChE catalyzes the hydrolysis of the neurotransmitter acetylcholine into choline and acetic acid, a reaction necessary to allow a cholinergic neuron to return to its resting state after activation. The inhibition of acetylcholinesterase (AChE) can contribute to increasing ACh brain levels.

Evaluation of Protection from H2O2-Induced Oxidative Stress:

We examined the neuroprotective effects of berberis extract against hydrogen peroxide in ARPE (retinal pigment epithelium) cells. The neuroprotective ability of the berberis extract was evaluated in ARPE cells induced with oxidative stress using hydrogen peroxide.

Oxidative stress contributes to the pathogenesis of acute or chronic neurodegenerative processes. The main cytotoxic reactive oxygen species are hydrogen peroxide and free radicals. The overproduction of hydrogen peroxide is associated with amyloid aggregation. The excessive production of free radicals damages cellular macromolecules such as lipids, proteins, and DNA, which results in mitochondrial and nuclear damage and induction of apoptosis in neuronal cells.

### Results of Biochemical Parameters:

| | AchE inhibition | Protection from |
|---|---|---|
| | % Inhibition | oxidative stress |
| Product | (IC50 μg/ml) | (% survival) |
| Berberis aristata | 90.2 (8.9) | 87.5 |
| extract/composition | | |

### Abbreviations

DTNB—Dithiobis (2-nitrobenzoic acid)
p.o—per oral
i.p—intraperitoneal
AST—Aspartate aminotransferase
ALT—Alanine transaminase
BUN—Blood urea nitrogen
CNS—Central nervous system
SGPT—serum glutamic pyruvic transaminase or Alanine amino transferase
SGOT—serum glutamic oxaloacetic transaminase or aspartate transaminase

We claim:

1. A composition comprising:
i) Berberis aristata extract, wherein the extract comprises Berberine hydrochloride in a range of 5% to 50% w/w of the extract; and
ii) at least one or more pharmaceutically or nutraceutically acceptable ingredients selected from the group consisting of hydrophilic carrier, PGP Inhibitor, Bioenhancer, Binder, emulsifier, pH modifier, and anticaking agent.

2. The composition according to claim 1, wherein the hydrophilic carrier is present in a range of 30% to 90% w/w of the composition and is selected from the group consisting of hydroxypropyl methyl cellulose, microcrystalline cellulose, polyacrylates, polyethylene glycols, povidones, starch and starch derivatives, gums, sugars, plant polysaccharides, and a combination thereof.

3. The composition according to claim 1, wherein the PGP Inhibitor is present in a range of 0.1% to 20% w/w of the composition and is selected from the group consisting of Piperine, Vitamin E TPGS (d-α-Tocopheryl polyethylene glycol 1000 succinate), alkaloids, polysorbates flavonoids, coumarins, resins, saponins, and a combination thereof.

4. The composition according to claim 1, wherein the Bioenhancer is present in a range of 0.01% to 10% w/w of the composition and is selected from the group consisting of piperine, d-limonene, monoglycerides, and phospholipids such as lecithin/phosphatidyl choline, glyceryl monostearate botanical extracts, and a combination thereof.

5. The composition as according to claim 1, wherein the binder is present in a range of 0.1% to 15% w/w of the composition and is selected from the group consisting of Hydroxy propyl methyl cellulose, Methyl cellulose, Hydroxy Propyl cellulose, Polyvinyl Pyrrolidone, Starch, and natural gums such as gum acacia, gum tragacanth, starch, guar gum, and a combination thereof.

6. The composition according to claim 1, wherein the emulsifier is present in a range of 0.01% to 10% w/w of the composition and is selected from the group consisting of stearic acid, phosphorylcholine, polysorbate, sugar alcohols, glycerol & derivatives, and a combination thereof.

7. The composition according to claim 1, wherein the pH modifier is present in a range of 0.01% to 10% w/w of the composition and is selected from the group consisting of sodium bicarbonate, citric acid, trisodium citrate, lactic acid, L-arginine, calcium carbonate, magnesium carbonate, and a combination thereof.

8. The composition according to claim 1, wherein the anticaking agent is present in a range of 0.01% to 5% w/w of the composition and is selected from the group consisting of silicon dioxide, magnesium stearate, stearic acid, mannitol, and a combination thereof.

9. The composition according to claim 1, the composition comprising:
i) Berberis aristata extract in an amount ranging from 20% to 50% w/w of the composition, wherein the extract comprises Berberine hydrochloride in an amount of 10% to 20% w/w of the extract,
ii) Microcrystalline Cellulose (MCC 101) in an amount of 62% to 68.5% w/w of the composition,
iii) Colloidal Silicon Dioxode (Aerosil) in an amount of 0.5% w/w of the composition,
iv) Sunflower Lecithin (Phosphatidylcholine) in an amount of 1% w/w of the composition,
v) Polysorbate 80 in an amount of 2% w/w of the composition,
vi) Calcium Carbonate in an amount of 4% w/w of the composition,
vii) Hydroxy propyl methyl cellulose (HPMC E3) in an amount of 1.5% w/w of the composition, and
viii) Methacrylic acid ethyl acrylate copolymer (Kollicoat MAE 30DP) in an amount of 2.5% w/w of the composition.

10. A method for preventing, improving, and/or maintaining cognitive health in a subject, comprising administering the composition according to claim 1 to a subject in need thereof.

11. A method for improving memory and recall, improving concentration and/or focus, improving cognitive skills such reasoning and/or decision making, improving daily productivity, multi-tasking, alertness, and/or attention, and/or managing stress and/or increase levels of BDNF in a subject, comprising administering the composition according to claim 1 to a subject in need thereof.

12. The method according to claim 10, wherein the subject is human and/or animal.

13. The composition according to claim 1, wherein the composition is in a dose form comprising 1 mg/kg to 200 mg/kg of body weight of subjects.

14. A process for the preparation of the composition according to claim 1, wherein the process comprises the following steps:
a) preparing a solution of Bioenhancer, Binder, PGP Inhibitor, and, emulsifier,
b) preparing a solution of Polymer,
c) adding berberis extract, pH modifier, and anticaking agent, and stirred to form a uniform blend,
d) mixing solutions prepared in steps (a) and (b) in the dry blend of step (c) to obtain the wet granules, and
e) drying and sifting the granules obtained in step (d).
